(19) United States
(12) Patent Application Publication
(10) Pub. No.: US 2019/0358196 A1
(43) Pub. Date: Nov. 28, 2019

(54) PI 4-KINASE INHIBITOR AS A THERAPEUTIC FOR VIRAL HEPATITIS, CANCER, MALARIA. AUTOIMMUNE DISORDERS AND INFLAMMATION, AND A RADIOSENSITIZER AND IMMUNOSUPPRESSANT

(71) Applicants: Henry C. Lowe, Kingston 5, West Indies (JM); Ngeh J. Toyang, Columbia, MD (US)

(72) Inventors: Henry C. Lowe, Kingston 5, West Indies (JM); Ngeh J. Toyang, Columbia, MD (US)

(21) Appl. No.: 16/321,333

(22) PCT Filed: Jul. 27, 2017
(86) PCT No.: PCT/US17/44153

§ 371 Date: Mar. 19, 2019

Related U.S. Application Data
(60) Provisional application No. 62/367,345, filed on Jul. 27, 2016.

(51) Int. Cl. A61K 31/353; A61K 41/00; A61P 31/14

(52) U.S. Cl. CPC A61K 31/353; A61P 31/14; A61K 41/0038

(57) ABSTRACT

The present invention provides a plant-based flavonoid pharmaceutical composition and its synthetic for inhibition of phosphau'dylinositol-4-kinases and consequent prevention and treatment of RNA viruses including but not limited to viral hepatitis, as well as activity against cancer, malaria, autoimmune disorders and inflammation, prevent organ transplant rejection and as a radiation sensitizer. A method for isolating specific plant-based flavonoid pharmaceutical compositions from raw plant material as well as a method for synthesizing the compositions are also disclosed.

### CROSS-REFERENCE TO RELATED APPLICATION(S)

The present application derives priority from U.S. Provisional Patent Application 62/367,345 filed 27 Jul. 2016, the entirety of which is incorporated herein by reference.

### BACKGROUND OF THE INVENTION

### 1. Field of the Invention

The present invention relates to flavonoid derivatives and, more particularly, to plant flavonoid derivatives or the pharmaceutically acceptable salt thereof that may be used in a pharmaceutical composition for preventing and treating viral hepatitis, cancer, autoimmune disorders and inflammation, to prevent organ transplant rejection, and as a radiosensitizer.

### 2. Description of the Background

Hepatitis C virus (HCV) infection is a major cause of chronic hepatitis, liver cirrhosis and hepatocellular carcinoma affecting millions of people worldwide. Hanafiah, Groeger, Flaxman, Wiersman, Global Epidemiology Of Hepatitis C Virus Infection: New Estimates Of Age-Specific Antibody To HCV Seroprevalence, ST Hepatology, April 57(4):1333-42 (2013). According to most recent statistics from the World Health Organization (WHO) (WHO Fact Sheet No 164, April 2014) the global burden of HCV is as follows:
130-150 million people globally have chronic hepatitis C infection.
A significant number of those who are chronically infected will develop liver cirrhosis or liver cancer.
350,000 to 500,000 people die each year from hepatitis C-related liver diseases.
Antiviral medicines can cure hepatitis C infection, but access to diagnosis and treatment is low as effective drugs are very expensive and out of the reach of many especially in developing countries.
Antiviral treatment is successful in 50-90% of persons treated, depending on the treatment used, and has also been shown to reduce the development of liver cancer and cirrhosis.

The discovery and development of effective and affordable treatments for HCV infections remains an important research objective. The recent discovery and development of new anti HCV agents with significantly higher efficacy than the interferon (IFN) and ribavirin (RBV) regimens has improved the treatment of HCV. Liang and Ghany, Current And Future Therapies For Hepatitis C Virus Infection, N Engl J Med. May 16; 368 (20): 1907-17 (2013).

Unfortunately, the efficacies of new direct-acting antivirals (DAAs) is unknown in some groups of patients with different subtypes of the virus as well as those with advance cirrhosis. See, Hanafiah et al., 2013, supra. In other to continue the search for new antiviral agents against HCV, attention has recently been directed towards discovering molecules that target host proteins or enzymes that play significant roles in the HCV life cycle. Salloum S and Tai A W, Treating Hepatitis C Infection By Targeting The Host., Transl Res 159:421-429 (2012). This approach is complementary to the DAA alternative as because like all viruses, HCV is an obligate parasite requiring a host cell for its own replication. See, Salloum et al., supra, (2012).

Among the several host factors responsible for HCV entry and replication in humans are the phosphatidylinositol-4-kinases. Tai A W, Benita Y, Peng L F, Kim S S, Sakamoto N, Xavier R J, Chung R T., A Functional Genomic Screen Identifies Cellular Cofactors Of Hepatitis C Virus Replication, Cell host & microbe, 2009; 5:29&-307; Li Q, Brass A L, Ng A, Hu Z, Xavier R J, Liang T J, Elledge S J, A Genome-Wide Genetic Screen For Host Factors Required For Hepatitis C Virus Propagation, Proc Natl Acad Sci USA, 2009; 106:16410-16415; Vaillancourt F H, Pilote L, Cartier M, Lippens J, Liuzzi M, Bethell R C, Cordingley M G, Kukolj G., Identification Of A Lipid Kinase As A Host Factor Involved In Hepatitis C Virus RNA Replication, Virology. 2009; 387:5-10; Borawski J, Troke P, Puyang X, Gibaja V, Zhao S, Mickanin C, Leighton-Davies J, Wilson C J, Myer V, Comellataracido I, et al., Class III Phosphatidylinositol 4-Kinase Alpha And Beta Are Novel Host Factor Regulators Of Hepatitis C Virus Replication, Journal of virology, 2009; 83:10058-10074; Reis, H. T., Maniaci, M. R., Caprariello, P. A., Eastwick, P. W., & Finkel, E. J., Familiarity Does Indeed Promote Attraction In Live Interaction, Journal of Personality and Social Psychology, 101, 557-570 (2011).

The family of PI4-kinases is made up of two types with two isoforms each (PI4KIIa, PI4KIIb, PI4KIIIa and PI4KIIIb) differing in subcellular localization and being responsible for the synthesis of distinct PI4P pools. Balla A., Balla T., Phosphatidylinositol 4-Kinases: Old Enzymes With Emerging Functions, Trends Cell Biol. 16:351-361 10.1016 (2006). In case of PI4KIIIa those in the ER, the plasma membrane and pans of Golgi PI4P. Balla A., Tuymetova G., Tsiomenko A., Várnai P., Balla T., A Plasma Membrane Pool Of Phosphatidylinositol 4-Phosphate Is Generated By Phosphatidylinositol 4-Kinase Type-III Alpha: Studies With The PH Domains Of The Oxysterol Binding Protein And FAPP1, Mol. Biol. Cell. 16:1282-1295 (2005); Bianco A., Reghellin V., Donnici L., Fenu S., Alvarez R., Baruffa C., Peri F., Pagani M., Abrignani S., Neddermann P., De Francesco R., Metabolism Of Phosphatidylinositol 4-Kinase liiα-Dependent PI4P Is Subverted By HCV And Is Targeted By A 4-Anilino Quinazoline With Antiviral Activity., PLoS Pathog. 8:e1002576 10.137/journal.ppat.1002576 (2012). PI4KIIIa has been identified as an essential host factor of HCV RNA replication by a number of studies. Berger K. L., Cooper J. D., Heaton N. S., Yoon R., Oakland T. E., Jordan T. X., Mateu G., Grakoui A., Randall G., Roles For Endocytic Trafficking And Phosphatidylinositol 4-Kinase III Alpha In Hepatitis C Virus Replication, Proc. Natl. Acad. Sci. USA. 106:7577-7582 (2009); Trotard M, Lepere-Douard C, Regeard M, Piquet-Pellorce C, Lavillette D, Cosset F L, Gripon P, Le Seyec J., Kinases Required In Hepatitis C Virus Entry And Replication Highlighted By Small Interference RNA Screening, FASEB. J., 23:3780-3789 (2009). See, also, Tai et al. (2009), supra, and Vaillancourt et al., (2009), supra.

The involvement of PI4KIII has also been reported as well, but might be restricted to genotype 1. Interestingly PI4KIII and PI4P are also closely linked to replication of enteroviruses, suggesting that dependence on PI metabolism and particularly PI4P is a common theme for many virus groups and suggesting the possibility that inhibitors of PI4KIII and PI4P may be broad acting antivirals agents.

PI4K kinases are also implicated in cancer onset and progression. PI4KIIIα and PI4KIIIβ have been linked to drug resistance and antiapoptotic effect in pancreatic and breast cancers respectively. V. Giroux, J. Iovanna, J. C. Dagorn, Probing the Human Kinome for Kinases Involved in Pancreatic Cancer Cell Survival and Gemcitabine Resistance, FASEB J. 20 1982-1991 (2006); K. Chu, S. Minogue, J. Hsuan, M. Waugh, Differential Effects of the Phosphatidylinositol 4-Kinases, PI4KIIIalpha And PI4KIIIbeta, oin Akt Activation And Apoptosis, Cell Death Dis. (2010) I; V. A. Tomlinson, H. J. Newbery, N. R. Wray, J. Jackson, A. Larionov, W. R. Miller, et al., Translation Elongation Factor Eef1a2 is a Potential Oncoprotein That is Overexpressed In Two-Thirds Of Breast Tumours, BMC Cancer 5 (2005) 113; A. A. Morrow, et al. The lipid kinase PI4KIIIbeta is highly expressed in breast tumors and activates Akt in cooperation with Rab11a, Mol. Cancer Res., 12 (2014), pp. 1492-1508.

PI4K kinases are also for the synthesis of PI4P which is responsible for cell proliferation and migration. Inhibition of PI 4-kinase activity as such could potentially provide a valuable therapeutic target for combined inhibition of both the PLC and PI 3-kinase pathways through limiting the supply of PI4P and PI(4,5)P2 during receptor-activated signalling. The PI3-kinases pathway is known for its role in cancer onset and progression. T. L. Yuan, L. C. Cantley, PI3K Pathway Alterations In Cancer: Variations On A Theme, Oncogene 27, 5497-5510 (2008); A. Balla, T. Balla, Phosphatidylinositol 4-kinases: Old Enzymes With Emerging Functions, Trends Cell Biol. 16, 351-361 (2006).

Inhibition of PI4K kinases have also been shown to have beneficial effects in treating autoimmune disorder and inflammation as well the prevention of cell and organ transplant rejection. Loo, L., Wright, B. D. and Zylka, M J., 2015. Lipid kinases as therapeutic targets for chronic pain. Pain, 156(0 1), p. S2; Herman, Jean, Louat, Thierry, Huang, Qiuya, Vanderhoydonck, Bart, Waer, Mark, Herdewijn, Piet (2014), Autoimmune and Inflammatory Disorder Therapy, United States Patent Application 20140294870.

Mutations in the PI4K kinase have also recently been found to be responsible for the development of resistant to chemotherapy by antimalarial medication and is as such seen as a target for the control of drug resistant Plasmodium parasite. Plasmodium's life cycle consists of several distinct stages as mosquito-injected sporozoites rapidly populate liver cells, in which they either proliferate and produce merozoites that emerge in the bloodstream or enter a dormant phase as hypnozoites in the liver. The life cycle is regulated by several cellular factors including the PI4KIIIβ kinase. McNamara et al., 2013 reported that the PI4KIIIβ is involved in all stages of the life cycle of the Plasmodium parasite and its inhibition haled the progression of parasite making this PI4KIIIβ a major therapeutic target against malaria. McNamara, C. W., Lee, M. C., Lim, C. S., Lim, S. H., Roland, J., Simon, O., . . . & Zeeman, A. M., Targeting Plasmodium Phosphatidylinositol 4-Kinase To Eliminate Malaria, Nature, 504 (7479)(2013), 248; Rajkhowa, S., Borah, S. M., Jha, A. N., & Deka, R. C. (2017). Design of Plasmodium falciparum PI (4) KIIIβ Inhibitor using Molecular Dynamics and Molecular Docking Methods. Chemistry Select, 2(5), 1783-1792; Rutaganira, F. U., Fowler, M. L., McPhail, J. A., Gelman, M. A., Nguyen, K., Xiong, A. Burke, J. E., Design And Structural Characterization Of Potent And Selective Inhibitors Of Phosphatidylinositol 4 Kinase, IIIβ, Journal of medicinal chemistry, 59(5), 1830-1839 (2016); Ren, J. X., Gao, N. N., Cao, X. S., Hu, Q. A., & Xie, Y, Homology Modeling And Virtual Screening For Inhibitors Of Lipid Kinase PI (4) K From Plasmodium, Biomedicine & Pharmacotherapy, 83, 798-808 (2016).

The above examples confirms that there is a plethora of evidence that PI4-kinases are potential therapeutic targets and their inhibitors alone or in combination with other direct-acting antiviral and antimalarial agents could play a significant role in the control of the hepatitis C virus and malaria in addition to other indications and conditions including but not limited to cancer, autoimmune disorders and inflammation, to prevent organ transplant rejection, and as a radiosensitizer.

Flavonoids are common constituents of plants and cover a wide range of functions including acting as yellow pigments in petals and leaves to attract pollinating insects. They might also appear as bluish pigments (anthocyanins) to receive certain wavelengths of light, which permits the plant to be aware of the photoperiod. Many of these flavonoids also protect the plants by being involved in the filtering of harmful ultraviolet light Some flavonoids play crucial roles in establishing symbiotic fungi, while at the same time they fight infections caused by pathogenic fungi.

Flavonoids have relevant pharmacological activities such as; antioxidant, antidiabetic, anti-inflammatory, antiallergic, antibiotic, antidiarrheal, CNS and against cancer. In particular administration of anthocyanoside oligomer appeared to improve subjective symptoms and objective contrast sensitivity in myopia subjects. Lee, J., Lee, H. K., Kim, C. Y., Hong, Y. J., Choe, C. M., You, T. W., & Seong, G. J., Purified High-Dose Anthocyanoside Oligomer Administration Improves Nocturnal Vision and Clinical Symptoms in Myopia Subjects, Br J Nutr., June; 93(6): 895-9 (2005).

Given the abundance of evidence supporting the health benefits of flavonoids, the present inventors have successfully isolated a very bioactive flavonoid from a supercritical fluid extract (SFE) of Vernonia acuminata, a plant from the Blue Mountains of Jamaica. The molecule has shown activity against hepatitis C virus (HCV) in-vitro and against a select number of cancer cell lines, viral hepatitis, malaria, autoimmune disorders and inflammation, and is suitable for use as a radiation sensitizer (“radiosensitizer”) and to prevent organ transplant rejection.

Apart from the direct-acting activity against the HCV virus, the flavonoid has demonstrated significant inhibitory activity against Class III phosphatidylinositol 4-kinases (PI4KA and PI14KB). The present invention relates to the use of the newly isolated flavonoids alone or in combination with other flavonoids or related bioactive compounds to treat or prevent viral hepatitis, malaria, cancer, autoimmune disorders and inflammation, and for use as a radiation sensitizer (“radiosensitizer”) and to prevent organ transplant rejection.

### SUMMARY OF THE INVENTION

It is, therefore, an object of the invention to provide a therapeutic flavonoid composition alone or in combination with other direct-acting antiviral and antimalarial agents for inhibition of phosphatidylinositol-4-kinases and consequent prevention and treatment of RNA viruses including but not limited to hepatitis, as well as treatment against cancer, malaria, autoimmune disorders and inflammation, to prevent organ transplant rejection, and for use as a radiation sensitizer. It is another object to provide a method for isolating specific plant-based flavonoid pharmaceutical compositions from raw plant material that are biologically active in the prevention and treatment of RNA viruses including but not limited to hepatitis, viral hepatitis, cancer, malaria, autoimmune disorders and inflammation, as a prophylactic to prevent organ transplant rejection, and as a radiation sensitizer. In accordance with the foregoing objects, the present invention provides a flavonoid-based pharmaceutical composition for the prevention and treatment of RNA viruses including but not limited to viral hepatitis, cancer, malaria, autoimmune disorders and inflammation, to prevent organ transplant rejection, and as a radiation sensitizer (“radiosensitizer”). The flavonoid-based pharmaceutical composition has a structure of the general formula of FIG. 1 or a pharmaceutically acceptable salt thereof

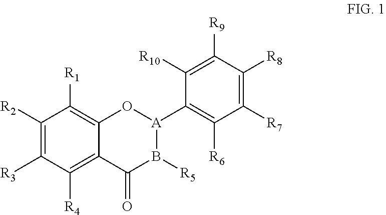

Wherein,

R1-R10 may be any one or more substituents selected from the group consisting of a hydrogen molecule (H), a hydroxide molecule (OH), a methyl group comprising one carbon atom bonded to three hydrogen atoms (CH3), an alkoxy group (O—CH3), a carboxyl group (COOH), chlorine (Cl), Bromine (Br), Fluorine (F), Glutamic acid (Glu), PEG chain and any salts or derivatives of the foregoing. A and B may be linked either by a single or double bond.

A method for isolating the specific plant-based flavonoid pharmaceutical compositions from raw plant material is also disclosed, as well as a method for prevention and treatment of RNA viruses including but not limited to viral hepatitis, cancer, malaria, autoimmune disorders and inflammation, to prevent organ transplant rejection, and as a radiation sensitizer. A method of treatment using the specific plant-based flavonoid pharmaceutical compositions above is also disclosed.

### BRIEF DESCRIPTION OF THE DRAWINGS

Other objects, features, and advantages of the present invention will become more apparent from the following detailed description of the preferred embodiments and certain modifications thereof when taken together with the accompanying drawings in which:

FIG. 1 is an illustration of the general plant-based flavonoid pharmaceutical compositions according to the present invention.

FIG. 2 is the structure of the specific plant-based flavonoid pharmaceutical composition.

FIG. 3 is a graphical illustration of how the kinase inhibition assay works.

FIG. 4. Is a block diagram of a suitable isolation scheme.

FIG. 5 is a process diagram illustrating a suitable synthesis approach.

### DETAILED DESCRIPTION OF THE PREFERRED EMBODIMENTS

Reference will now be made in detail to preferred embodiment of the present invention, examples of which are illustrated in the accompanying drawing.

The present invention is a group of plant-based flavonoid pharmaceutical compositions isolated from a supercritical fluid extract (SFE) of Vernonia acuminata, a plant from the Blue Mountains of Jamaica, and useful for the prevention and treatment of RNA viruses including but not limited to viral hepatitis, cancer, malaria, autoimmune disorders and inflammation, as a prophylactic to prevent organ transplant rejection and as a radiation sensitizer (“radiosensitizer”).

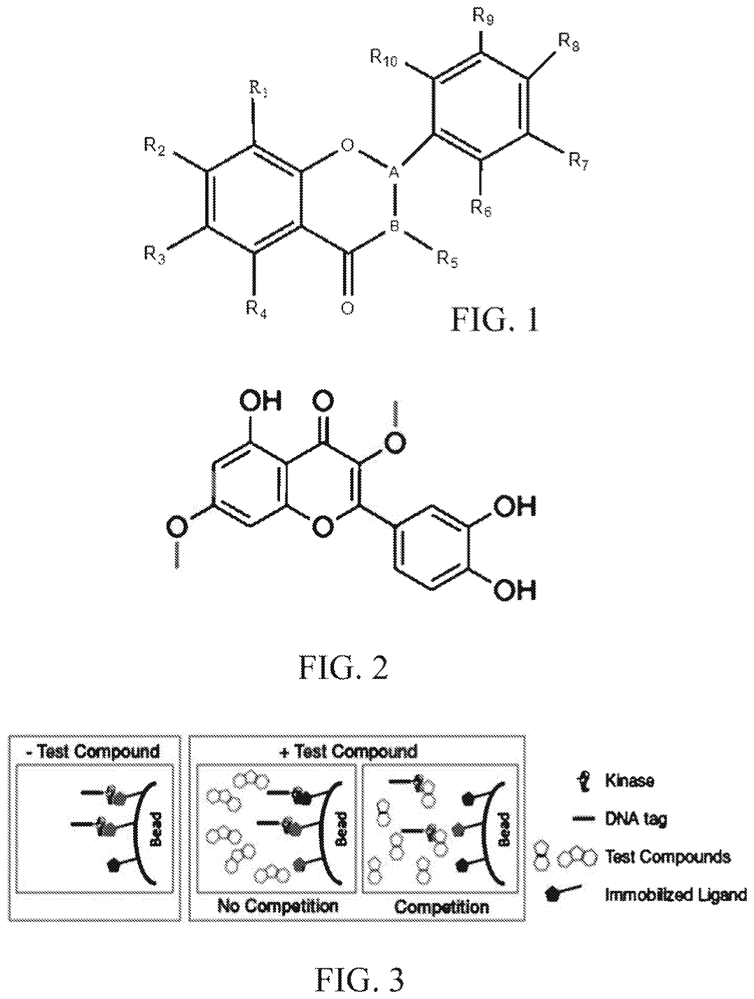

The plant-based flavonoid pharmaceutical composition for the prevention and treatment of RNA viruses including but not limited to hepatitis, intracellular bacteria and malaria has the structure of the general formula of FIG. 1 or a pharmaceutically acceptable salt thereof.

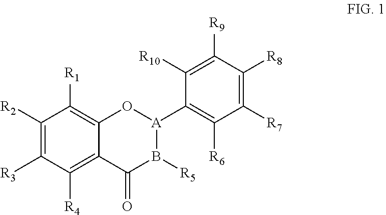

Wherein,

R1-R10 may be any one or more substituents selected from the group consisting of a hydrogen molecule (H), a hydroxide molecule (OH), a methyl group comprising one carbon atom bonded to three hydrogen atoms (CH3), an alkoxy group (O—CH3), a carboxyl group (COOH), chlorine (Cl), Bromine (Br), Fluorine (F), Glutamic acid (Glu), and any salts or derivatives of the foregoing. A and B may be linked either by a single or double bond.

The most preferred structure of the synthesized flavonoid presented in FIG. 2.

In an embodiment, a method for the prevention and treatment of RNA viruses including but not limited to hepatitis, intracellular bacteria and malaria using the specific plant-based flavonoid pharmaceutical compositions above is also disclosed. Administration may be by various routes including oral, rectal or intravenous, epidural muscle, subcutaneous, intrauterine, or blood vessels in the brain (intracerebroventricular) injections. The flavonoid derivatives of the general and specific formulas (FIGS. 1-2) according to the present invention and a pharmaceutically acceptable salt thereof may be administered in an effective dose, depending on the patient's condition and body weight, extent of disease, drug form, route of administration, and duration, within a range of from 0.1 to 500 mg between 1-6 times a day. Of course, most dosages will be by a carrier. The specific dose level and carrier for patients can be changed according to the patient's weight, age, gender, health status, diet, time of administration, method of administration, rate of excretion, and the severity of disease.

The composition may be formulated for external topical application, oral dosage such as powders, granules, tablets, capsules, suspensions, emulsions, syrups, aerosols, suppositories, or in the form of a sterile injectable solution. Acceptable carriers and excipients may comprise lactose, dextrose, sucrose, sorbitol, mannitol, xylitol, erythritol, maltitol, starches, gum acacia, alginate, gelatin, calcium phosphate, calcium silicate, cellulose, methyl cellulose, microcrystalline cellulose, polyvinylpyrrolidone, water, methyl benzoate, propyl benzoate, talc, magnesium stearate, polyethylene glycol and mineral oil.

Bioactivity of the above-described compounds have been verified by use of kinase inhibition assays to determine the effect of the flavonoids in the onset and progression of RNA viruses and cancer. The inhibition of PI4K kinases in particular has been shown to be a therapeutic target that could block the replication of RNA viruses including but not limited to viral hepatitis, as well as cancer, malaria, autoimmune disorders and inflammation, organ transplant rejection and as a radio-sensitizer.

### Anti Hepatitis C Activity

Huh7.5 cells are grown in Dulbecco's modified essential media (DMEM), 10% fetal bovine serum (FBS), 1% penicillin-streptomycin (pen-strep), 1% Non-essential amino acids (NEAA) in a 5% CO₂ incubator at 37° C. Huh7.5 cells will be seeded at 1×10⁴ cells per well into 96-well plates according to Southern Research Institute standard format. Test article will be serially diluted with DMEM plus 5% FBS. The diluted compound in the amount of 50 μl will be mixed with equal volume of cell culture-derived HCV (HCVcc), then applied to appropriate wells in the plate. Human interferon alpha-2b (rIFNα-2b) is included as a positive control compound. After 72 hr incubation at 37° C., the cells were lysed for measurement of luciferase activity using Renilla Luciferase Assay System (Promega) according to manufacturer's instruction. The number of cells in each well will be determined by CytoTox-1 reagent (Promega). Test articles are tested with 6 serial dilution in triplicate to derive, if applicable, IC₅₀ and IC₉₀ (concentration inhibiting HCVcc infectivity by 50% and 90%, respectively), TC₅₀ (concentration decreasing cell viability by 50%) and SI (selective index: TC₅₀/IC₅₀) values.

Results of the inhibition of HCVcc are indicated in the table below

| Compound | Test Concentration | EC₅₀ | CC₅₀ | SI (CC₅₀/EC₅₀) |
|---|---|---|---|---|
| rIFNa-2b | 10 IU/mL | 0.63 | >10.0 | >15.9 |
| FBL-02 | 100 μg/mL | 1.37 | 4.18 | 3.05 |

Kinase Inhibition Assay

In vitro profiling of 12 lipid kinase was accomplished using the “HotSpot” assay platform. Briefly, specific kinase/substrate pairs along with required cofactors were prepared in reaction buffer 20 mM Hepes pH 7.5, 10 mM MgCl2, 1 mM EGTA, 0.02% Brij35, 0.02 mg/ml BSA, 0.1 mM Na3VO4, 2 mM DTT, 1% DMSO. Compounds were delivered into the reaction, followed ˜20 min later by addition of a mixture of ATP (Sigma) and 33P ATP (PerkinElmer) to a final concentration of 10 μM. Reactions were carried out at 25° C. for 120 min, followed by spotting of the reactions onto P81 ion exchange filter paper (e.g., Whatman Ashless Filter Paper). Unbound phosphate was removed by extensive washing of filters in 0.75% phosphoric acid. After subtraction of background derived from control reactions containing inactive enzyme, kinase activity data were expressed as the percent remaining kinase activity in test samples compared to vehicle (dimethyl sulfoxide) reactions. IC₅₀ values and curve fits were obtained using Prism™ (by GraphPad Software). Kinome tree representations were prepared using Kinome Mapper.

To determine the kd values, competition binding assays were established, authenticated and executed as described previously (Fabian et al., 2005, Karaman et al., 2008). For most assays, kinases were fused to T7 phage strains (Fabian et al. 2005) and for the other assays, kinases were produced in HEK-293 cells after which they were tagged with DNA for quantitative PCR detection (data not shown). In general, full-length constructs were used for small, single domain kinases, and catalytic domain constructs for large multi-domain kinases. The binding assays utilized streptavidin-coated magnetic beads treated with biotinylated small molecule ligands for 30 minutes at room temperature which generated affinity resins for the kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific phage binding. Binding reactions were assembled by combining kinases, liganded affinity beads, and test compounds in 1× binding buffer (20% SeaBlock, 0.17×PBS, 0.05% Tween 20, 6 mM DTT). Test compounds were prepared as 40× stocks in 100% DMSO and diluted directly into the assay (Final DMSO concentration=2.5%). All reactions were performed in polypropylene 384-well plates in a final volume of 0.04 mi. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM non-biotinylated affinity ligand) and incubated at room temperature with shaking for 30 minutes. The kinase concentration in the eluates was measured by quantitative PCR. A graphical illustration of the kinase interaction process is presented below. Kd values were determined using a standard dose response curve using the hill equation. Curves were fitted using a non-linear least square fit with the Levenberg-Marquardt algorithm.

FIG. 3 is a graphical illustration of how the foregoing assay works.

Percent Control (% Ctrl)

The compound(s) were screened at 10 μM and results for primary screen binding interactions are reported as ‘% Ctrl’, where lower numbers indicate stronger hits in the matrix.

### % Ctrl Calculation

$$( \frac{test compound signal - positive control signal}{negative control signal - positive control signal} ) \times 100$$

test compound=compound submitted by Environmental Health Foundation
negative control=DMSO (100% Ctrl)
positive control=control compound (0% Ctrl)

Results of the inhibition of 12 lipid kinases by FBL-02 and its analogs

Are shown in the table below:

| | Compound IC50 (M) | | | | | | |
|---|---|---|---|---|---|---|---|
| Kinase: | FBL-02 | FBL-1023 | FBL-1074 | FBL-1305 | FBL-1136 | PI-103 | PIK-93 |
| PI3Ka | 5.16E−07 | <1.23E−06 | <1.23E−06 | | <1.23E−06 | 2.64E−09 | ND |
| PI3Kb | 2.27E−06 | 4.59E−06 | | | 1.05E−06 | 1.52E−09 | ND |
| PI3K | 5.76E−07 | <1.23E−06 | <1.23E−06 | | <1.23E−06 | 2.54E−09 | ND |
| (p110a(E542K)/p85a) | | | | | | | |
| PI3Kd | 1.34E−06 | <1.23E−06 | <1.23E−06 | | <1.23E−06 | 4.69E−09 | ND |
| PI3KC3 | 5.09E−07 | <1.23E−06 | <1.23E−06 | <1.23E−06 | <1.23E−06 | 1.50E−08 | 2.26E−08 |
| PI4Ka | 6.10E−06 | 2.78E−05 | | | 5.30E−06 | 3.22E−07 | 5.43E−08 |
| PI4Kb | 5.22E−09 | <1.23E−06 | <1.23E−06 | <1.23E−06 | <1.23E−06 | 1.96E−05 | 4.37E−09 |
| PI4K2A | 3.05E−06 | 6.87E−05 | | | 1.02E−06 | ND | 4.77E−05 |

Kd values of V. acuminata extract and FBL-02 against PI4Kcb kinase

| Sample | Kd (ng/ml) |
|---|---|
| V. acuminata extract | 230 |
| FBL-02 | 4.8 |

Anticancer activity assay.

CellTiter Glo Assay Cell Viability Assay.

Cells were seeded in 96 well plates, one for each cell line, and incubated overnight. The following day, cells were exposed to drug treatment and incubated for 72 h. At the end of the 72 h. exposure period, CellTiter Glo reagent was added to the wells for 2 mins, followed by a further 10 min incubation at room temperature. Cell viability was determined from

Luminescence readings and IC₅₀ extrapolated from dose response curves using GraphPad Prism™ software. The result of the activity of FBL-02 against 12 cancer cell lines is present in the table below.

| Type of Cancer | | IC₅₀ Values |
|---|---|---|
| Cancer | Cell Line | (μM) |
| Breast | MCF-7 | 7.4 |
| Colon | COLO-205 | 18 |
| Colon | DLD-1 | 7.8 |
| Kidney | A498 | 50 |
| Lung | A549 | 9.5 |
| Lung (Small) | NCI-H69 | 9.3 |
| Lymphoma | RL | 5.4 |
| Melanoma | UACC-62 | 20 |
| Ovarian | IGROV-1 | 7.1 |
| Pancreatic | CFPAC-1 | 5.9 |
| Pancreatic | MiaPaca-2 | 17 |
| Prostate | PC-3 | 11 |

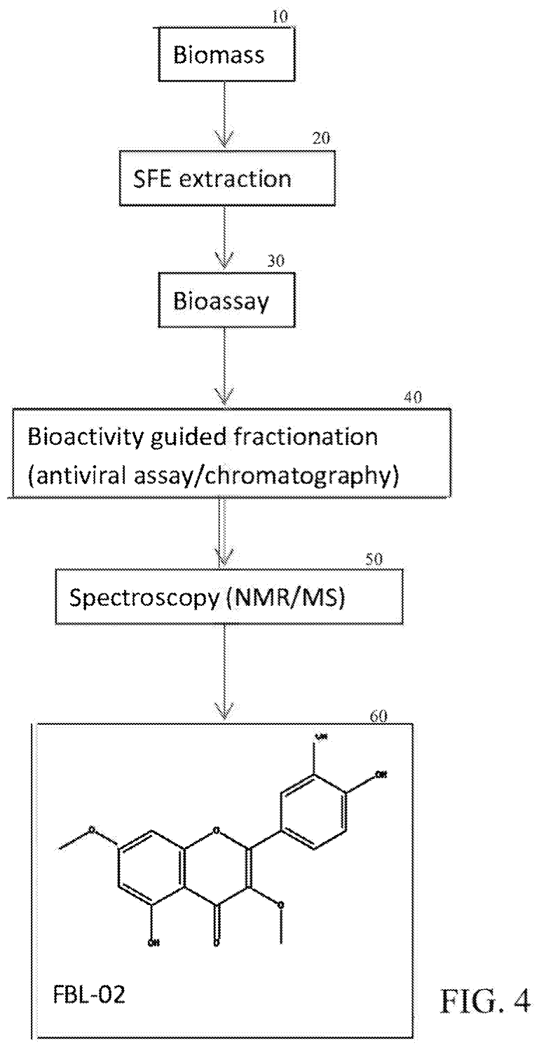

A method for isolating the specific flavonoid pharmaceutical compositions from raw plant material is also disclosed. The isolation was realized according to the scheme shown in FIG. 4.

At step 10 an appropriate amount of plant biomass is collected. For present purposes, Vernonia acuminate, a plant from the Blue Mountains of Jamaica, was collected by hand. The collected plant material was air dried under shade and pulverized into powder.

At step 20 the powder is subjected to supercritical fluid extraction (SFE) by which carbon dioxide (CO²) is used for separating one component (the extractant) from another (the matrix). The extract is evaporated to dryness resulting in a green residue.

At step 30, for experimental purposes, a bioassay-guided fractionation was employed, using a standard protocol to isolate a pure chemical agent from its natural origin. This entailed a step-by-step separation of extracted components based on differences in their physicochemical properties, and assessing all their biological activity. The extracted components may, for example, be fractionated by dry column flash chromatography on Si gel using hexane/CH2Cl2/ethyl acetate and mixtures of increasing polarity to yield different fractions. The sample is then degassed by ultra-sonication to yield an insoluble solid, which solid is then filtered. The sample may then be subjected to high performance liquid chromatography (HPLC) using a column Phenomenex Luna™ C18, 5 μm, 2×50 mm; eluent, acetonitrile with 0.05% MeOH to confirm the presence of the various fractions.

At step 40, bioactivity of the extracts were verified in a kinase inhibition assay as described above. This identified the bioactive flavonoids from all the supercritical fluid extracts (SFE). As reported previously, the identified plant-based flavonoid extracts showed activity against several kinases implicated in the pathogenesis of the prevention and treatment of RNA viruses including but not limited to hepatitis, intracellular bacteria and malaria.

The next step was to identify the plant-based flavonoid constituents responsible for the observed kinase inhibitory activities and to further isolate them.

At step 50 Nuclear Magnetic Resonance Spectroscopy and mass spectrometry (NMR/MS) was performed and the interpreted spectra were consistent with plant-based flavonoid compositions, as identified above, and as shown in step 60. The bioactive plant-based flavonoid extracts found bioactive for the prevention and treatment of RNA viruses had the structure of the general formula of FIG. 1, and the specific structure of FIG. 2.

The compound is designated FBL-02, and purity of the compound FBL-02 was confirmed by HPLC prior to spectroscopic analysis.

Given the known structure of the general formula of FIG. 1, a method for synthesizing the same becomes possible. The bioactive plant-based flavonoid pharmaceutical composition may be synthesized by the phenylpropanoid metabolic pathway in which the amino acid phenylalanine is used to produce 4-coumaroyl-CoA. The 4-coumaroyl-CoA is combined with malonyl-CoA to yield the flavonoid backbone, which contains two phenyl rings. From here conjugate ring-closure of chalcones results in the familiar form of flavonoids, the three-ringed structure of a flavone.

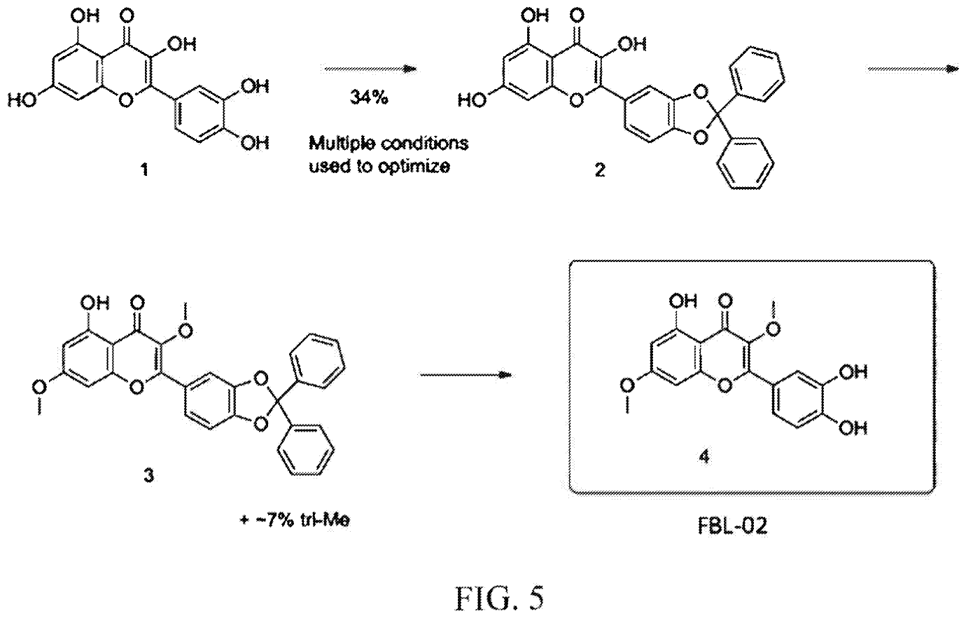

FIG. 5 is a process diagram illustrating a suitable synthesis approach. The metabolic pathway continues through a series of enzymatic modifications to yield the desired Flavone, Flavanone and Flavanol as identified above, and as shown in step 60. Of course, one skilled in the art will readily understand that other methods for synthesis are possible, such as the asymmetric methods set forth in Nibbs, A E; Scheidt, K A, “Asymmetric Methods for the Synthesis of Flavanones, Chromanones, and Azaflavanones”, European journal of organic chemistry, 449-462. doi:10.1002/ejoc.201101228, PMC 3412359, PMID 22876166 (2012).

It should now be apparent that the above-described invention provides a pharmaceutical composition for inhibition of phosphatidylinositol-4-kinases and consequent prevention and treatment of RNA viruses including but not limited to viral hepatitis, as well as cancer, malaria, autoimmune disorders and inflammation, to prevent organ transplant rejection and as a radiation sensitizer. The invention also provides a method for isolating the flavonoid pharmaceutical compositions from raw plant material.

It is to be understood, therefore, that the invention may be practiced otherwise than as specifically set forth in the appended claims.

### STATEMENT OF INDUSTRIAL APPLICABILITY

The family of PI4-kinases are linked to hepatitis C virus and malaria in addition to other indications and conditions including but not limited to cancer, autoimmune disorders and inflammation. There is evidence that PI4-kinases are potential therapeutic targets and their inhibitors, alone or in combination with other direct-acting antiviral and antimalarial agents, could play a significant role in the control of such indications and conditions. There would be great industrial applicability in a PI4-kinase inhibitor for therapeutic treatment of cancer, malaria, autoimmune disorders and inflammation, to prevent organ transplant rejection, and as a radiosensitizer.

We claim:

1. A method for the treatment of a patient in need thereof by administering to said patient a compound having a general chemical structure as shown below, or any pharmaceutically acceptable salt thereof: 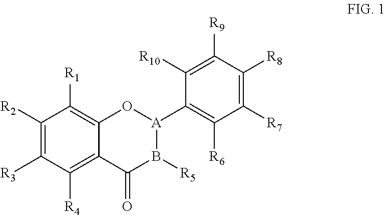
wherein,
R1-R10 may be any one or more substituents selected from the group consisting of a hydrogen molecule (H), a hydroxide molecule (OH), a methyl group comprising one carbon atom bonded to three hydrogen atoms (CH3), an alkoxy group (O—CH3), a carboxyl group (COOH), chlorine (Cl), Bromine (Br), Fluorine (F), Glutamic acid (Glu), and any salts or derivatives of the foregoing, and A and B may be linked by either a single or double bond.

2. The method according to claim 1 for the treatment of a patient having an RNA virus.

3. The method according to claim 2 for the treatment of a patient having viral hepatitis.

4. The method according to claim 1 for the treatment of a patient having malaria.

5. The method according to claim 1 for the treatment of a patient having cancer.

6. The method according to claim 1 for the treatment of a patient having an autoimmune disorder.

7. The method according to claim 1 for the amelioration of inflammation in said patient.

8. The method according to claim 1 for prophylactic prevention of organ transplant rejection by said patient.

9. The method according to claim 1 for use as a radiosensitizer during treatment of said patient.

10. A compound comprising a purified extract of the Vernonia acuminata plant or synthetic replica thereof having a general chemical structure as shown below, or any pharmaceutically acceptable salt thereof: 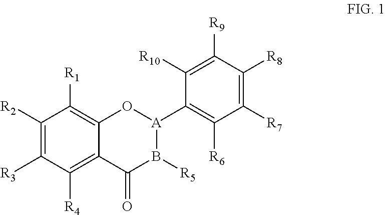
wherein,
R1-R10 may be any one or more substituents selected from the group consisting of a hydrogen molecule (H), a hydroxide molecule (OH), a methyl group comprising one carbon atom bonded to three hydrogen atoms (CH3), an alkoxy group (O—CH3), a carboxyl group (COOH), chlorine (Cl), Bromine (Br), Fluorine (F), Glutamic acid (Glu), and any salts or derivatives of the foregoing, and A and B may be linked by either a single or double bond.

11. The extract of claim 10, derived from said Vernonia acuminata plant by supercritical fluid extraction

12. A method of treating cancer, the method comprising administering the extract of claim 10.

13. The method of claim 12, wherein said extract is administered in a concentration within a range of from 0.1 to 500 mg between 1-6 times per day.

14. The method of claim 12, wherein said extract is administered in a formulation comprising a carrier, said carrier being selected from the group consisting of: lactose, dextrose, sucrose, sorbitol, mannitol, xylitol, erythritol, maltitol, starches, gum acacia, alginate, gelatin, calcium phosphate, calcium silicate, cellulose, methyl cellulose, microcrystalline cellulose, polyvinylpyrrolidone, water, methyl benzoate, propyl benzoate, talc, magnesium stearate, and mineral oil.

15. The method of claim 10, wherein the cancer treated by said extract is a type selected from the group comprising brain, breast, colon, kidney, leukemia, lung, lymphoma, melanoma, ovarian, pancreatic, and prostate cancers.

16. A method of treating inflammation, the method comprising:
administering the extract of claim 10.

17. The method of claim 16, wherein said extract is administered in a concentration within a range of from 0.1 to 500 mg between 1-6 times per day.

18. The method of claim 16, wherein said extract is administered in a form selected from the group consisting of: powders, granules, tablets, capsules, suspensions, emulsions, syrups, aerosols, and suppositories.

19. The method of claim 16, wherein said extract is administered in a formulation comprising a carrier, said carrier being selected from the group consisting of: lactose, dextrose, sucrose, sorbitol, mannitol, xylitol, erythritol, maltitol, starches, gum acacia, alginate, gelatin, calcium phosphate, calcium silicate, cellulose, methyl cellulose, microcrystalline cellulose, polyvinylpyrrolidone, water, methyl benzoate, propyl benzoate, talc, magnesium stearate, and mineral oil.

20. A method of treating viral hepatitis, the method comprising administering the compound of claim 10.

21. The method of claim 20, wherein said compound is administered in a concentration within a range of from 0.1 to 500 mg between 1-6 times per day.

22. A method of treating autoimmune disorders, the method comprising administering the compound of claim 10.

23. The method of claim 22, wherein said compound is administered in a concentration within a range of from 0.1 to 500 mg between 1-6 times per day.

24. A method of sensitizing tumor cells for radiation therapy, the method comprising:
administering the compound of claim 10.

25. The method of claim 24, wherein said compound is administered in a concentration within a range of from 0.1 to 500 mg between 1-6 times per day.

26. A method of preventing organ transplant rejection by administering the compound of claim 10.

27. The method of claim 26, wherein said compound is administered in a concentration within a range of from 0.1 to 500 mg between 1-6 times per day

28. A method of treating malaria, the method comprising administering the compound of claim 10.

29. The method of claim 28, wherein said compound is administered in a concentration within a range of from 0.1 to 500 mg between 1-6 times per day.
